(19) United States
(12) Patent Application Publication
(10) Pub. No.: US 2020/0124589 A1
(43) Pub. Date: Apr. 23, 2020

(54) Alcohol Testing Machine With Lock Controller

(71) Applicant: Yolanda Gonzales, Lafayette, CO (US)

(72) Inventor: Yolanda Gonzales, Lafayette, CO (US)

(21) Appl. No.: 16/591,550

(22) Filed: Oct. 2, 2019

Related U.S. Application Data
(60) Provisional application No. 62/742,159, filed on Oct. 5, 2018.

(51) Int. Cl. G01N 33/497; G07C 9/00; B60R 25/24; B60K 28/06

(52) U.S. Cl. CPC G01N 33/4972; G07C 9/00309; B60R 2325/101; B60K 28/06; B60W 2540/24; B60R 25/24

(57) ABSTRACT

A computer-based system controlling access to a locked vehicle uses an alcohol tester, receiving a user's breath, and producing an output indicative of an amount of alcohol in the user's breath. The signal is sent to a smartphone that runs an app that allows unlocking a wirelessly controlled lock, if the amount of alcohol in the user's breath is under a set limit.

### BACKGROUND

Devices are known for preventing people from driving automobiles while under the influence of alcohol. These devices are commonly known as interlocks. An interlock device is typically retrofitted into a vehicle and prevents the vehicle from being started and/or placed into drive mode unless a user breathes into the device. The device tests the alcohol level of the user from the breath, and determines whether to allow the vehicle to be driven.

These devices, however, are expensive to install, and require significant maintenance and inspection. Moreover, the interlocks are installed on a vehicle by vehicle basis, so that any user who uses a vehicle which has an interlock needs to breathe into the device, even when that user is not a high DUI risk person.

### SUMMARY OF THE INVENTION

The inventor recognized, however, that there are a number of drawbacks with the current systems.

As mentioned above, the systems are very expensive.

Moreover, many of these systems have little or no identification verification. A conventional “interlock” does nothing to ascertain that the person performing the breath test is actually the person will be driving. This makes it possible for individuals to falsify the test.

An aspect of the present invention is to provide a more simplified system which addresses many of these drawbacks.

An embodiment describes a system, referred to herein as “the AlcoBloc”, which operates wirelessly to enable protecting a vehicle against someone operating the vehicle while intoxicated.

In an embodiment, as described herein, a breath alcohol tester is described which is wireless enabled to control opening/unlocking of a wireless lock. A user operates the alcohol tester using a mobile application that runs, for example, on a smartphone. In one embodiment, the wireless operation can be by Bluetooth.

The lock is secured in a way that prevents the vehicle from being driven until the lock is opened. The lock can only be opened by a wireless command that can only be issued when the alcohol tester determines a breath which is free and clear of alcoholic content. In addition, a photo taken by the smart phone is correlated to the breath tester, to prevent fraudulent use of the system.

In an embodiment, the lock can be a padlock with a chain that is secured to an item on the vehicle. In an alternative embodiment, the lock can be a steering wheel lock of a conventional type typically used for antitheft purposes.

In embodiments, the lock can only be opened via the mobile app when the individual blows into the portable breath tester and either no alcohol is detected, or the amount of alcohol detected is below a specified level.

In an embodiment, individuals taking the test on the portable breath tester immediately have their picture taken by the mobile app of the smart phone upon initialization of the test. This picture can be taken by the mobile app at the time when the individual begins to blow into the device.

### BRIEF DESCRIPTION OF THE DRAWINGS

In the Drawings:

FIG. 1 shows the different components making up the invention;

FIG. 2 shows a block diagram of the alcohol tester and interface with the smart phone;

FIG. 3 shows a flowchart of operation; and

FIG. 4 shows an exemplary lock.

### DETAILED DESCRIPTION

The present application describes a system for use with preventing access to various resources by intoxicated users. An embodiment describes preventing access to a vehicle using the system. However it should be understood, as described herein, that this system can also be used for preventing access to various other devices and resources. One aspect of the present embodiment is the verification of the user's identity during testing to prevent an unauthorized user from carrying out the breath test and thus fooling the system in this way.

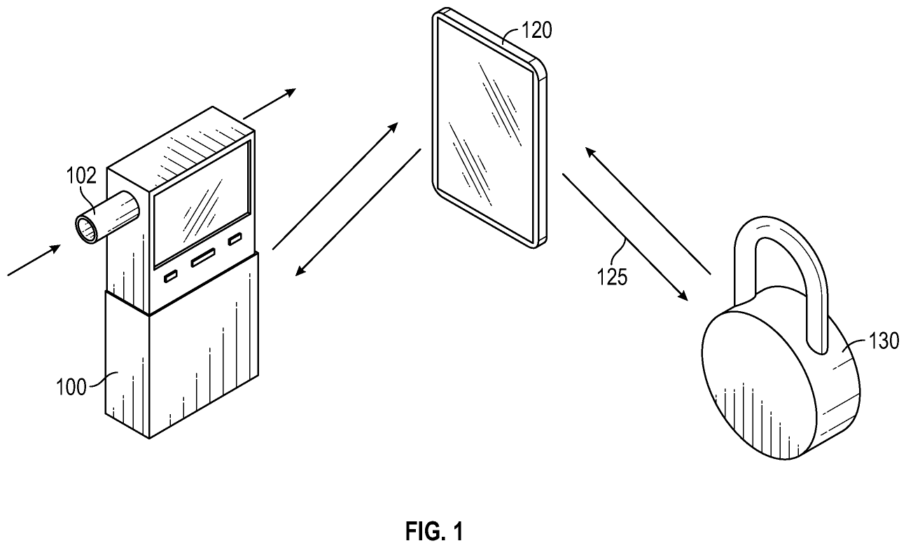

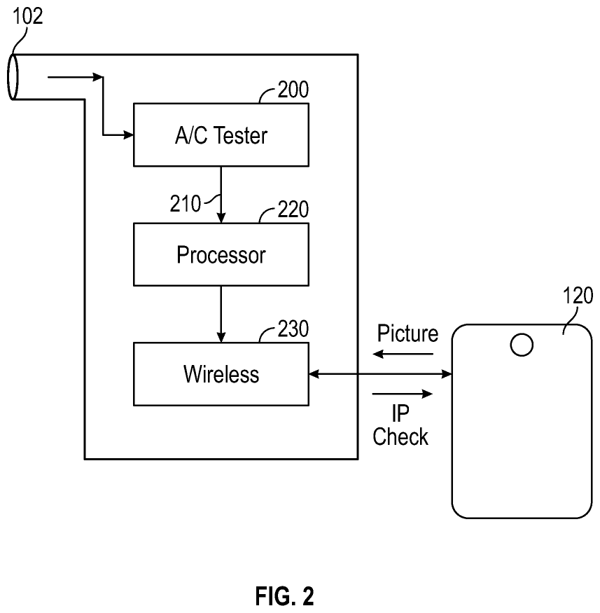

An embodiment describes a wireless tester, shown generally as 100 in FIG. 1. The wireless tester includes a tube 102 which, as conventional, is blown into by a user. In one embodiment, the tube can be covered by a disposable tube cover so that different users can use the device and all maintain sanitary conditions. In an alternative embodiment, the housing simply includes an opening without a tube. The breath into the unit passes through an alcohol testing part 200, shown in FIG. 2 which shows a block diagram of the alcohol tester of the present application. The alcohol tester detects an amount of alcohol in the breath and produces an output 210 of the alcohol tester which is indicative of the amount of alcohol in the breath.

The alcohol tester 200 can use any kind of conventional mobile alcohol tester as used in electronic testing systems. However, instead of displaying the number indicative of the alcohol output, this system produces an electronic output indicative of that numerical value.

For example, this can be a semiconductor oxide based tester which is an ethanol-specific tester that uses a tin oxide material to determine a percentage of ethanol in the subject's breath. Alternatively, this can be a fuel-cell tester, which provides more accurate readings of the alcohol content by creating a chemical reaction that oxidizes the alcohol in the breath sample and produces an electrical current indicative thereof. A greater current is produced by more alcohol being oxidized.

However, the alcohol tester 200 can be any of these systems or can be any other way of determining alcohol in a users breath. Any of these alcohol tester is 200, are referred to herein collectively as a breathalyzer.

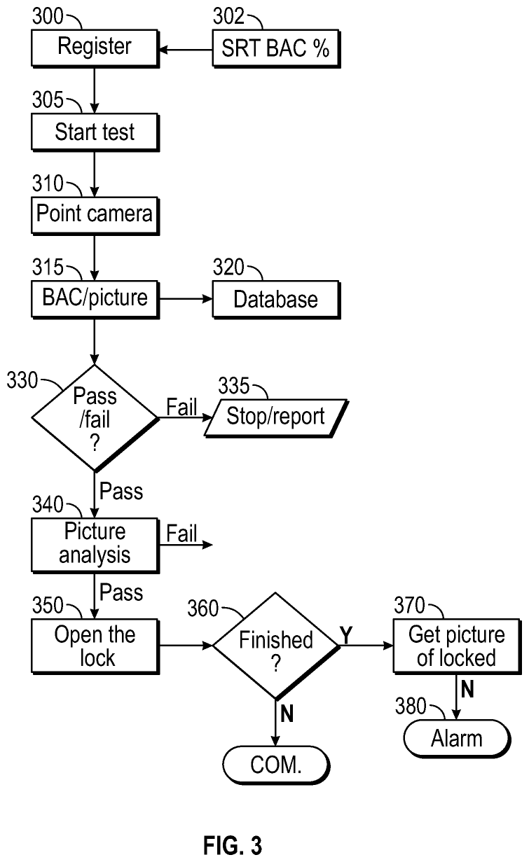

The signal output 210 of the alcohol tester is communicated via a wireless device to the smart phone 120. The smart phone 120 runs an application or “app”, which operates according to the flowchart of FIG. 3. The processor also communicates with a wireless device 230 which can use a wireless communication standard, such as, for example, Bluetooth or Wi-Fi The wireless device 230 can operate as described herein.

The processing of the operation can occur as shown in FIG. 3. This flowchart can be carried out by the smart phone 120 responsive to the breathalyzer 200 reporting the sensor value as a blood alcohol percentage to the smart phone 120. The parameters for the application on the Smart Phone uses are downloaded from a password protected online server. These parameters downloaded to the Smart Phone app may include information such as alcohol level pass/fail limits.

The parameters sent from the server to the Smart Phone app will be initially preset in a user account on the server. These parameters may be changed by allowing an approved individual to register onto this account and change the parameters used by the Smart Phone application.

At 300, an initial step is carried out where the user registers with the system. This may be a registration with a remote server, with a network of an organization whose resources are to be protected, or the like. In the case of a organization, this may protect access to the organization's vehicles or facilities.

After registering at 300, the system is ready for use. At 305, the user starts the test which will result in their obtaining access to the vehicle, by either initiating a button on the tester or on the smart phone, or simply by blowing into the tube 102.

The user is instructed at 310 to point the camera at the user while taking the test. At 315, the system acquires both the breath alcohol content (BAC) from the test, and also a picture of the user when carrying out the test. The system may alternatively acquire other information, such as the location at which the test is being taken (from the GPS of the smart phone), and other information as desired. All of this raw data can be stored in a database at 320, depending on user preferences and how much the user is willing to pay for.

The results may be sent to the online server for storage and review. In another embodiment, the results can be saved on the smart phone for a time, and/or may be sent to the online server.

At 330, processing of the breath alcohol level, which correlates to the blood alcohol level, is carried out to determine whether the breath tests passes or fails.

In this embodiment, a threshold for passing the breath alcohol test can be set at 302. For example, this can be set at a value which is high enough to avoid false readings, such as 0.01 or 0.02. If the test fails, this means that the detected breath alcohol is over the value set in 302. The system reports wirelessly e.g. to an administrator and/or to a database, that the user has failed the breath alcohol test and the lock signal remains closed.

If the test is passed at 330, then at 340, the picture may be stored, in an embodiment. This enables storing photo evidence of the person who actually took the test, in case there is a later question of who took this test. Other data can also be stored, e.g., time, date, location or other test parameters.

Also in one embodiment, the results can be emailed, e.g., to an administrator. A photo is taken at all times when the testing is done. The photo provides evidence if needed later on.

As an alternative, the picture analysis 340 can use “face identification”, which is a biometric test based on a photo of an authorized face, to determine if the picture matches another picture that is on file.

If the picture analysis passes, then a signal is sent to open the lock at 350. A picture analysis that fails can prompt the user to return to take a new picture, or can request a manual review of the picture for example by a manual reviewer.

In an alternative embodiment, the tester is automatically activated by the pressure of breath air flow into the device and immediate starting of the test is carried out at 305.

Embodiments can automatically unlock the wireless controlled lock when the user properly passes the BAC test, by electronically unlocking the lock 130 shown in FIG. 1. 130 in FIG. 1 shows a battery-operated wireless controllable lock 130 which is opened by a encrypted wireless signal 125 which is sent from the smart phone 120 after the app determines that the user has passed the breath test.

In an alternative embodiment, the encrypted wireless signal can be sent from the alcohol tester itself.

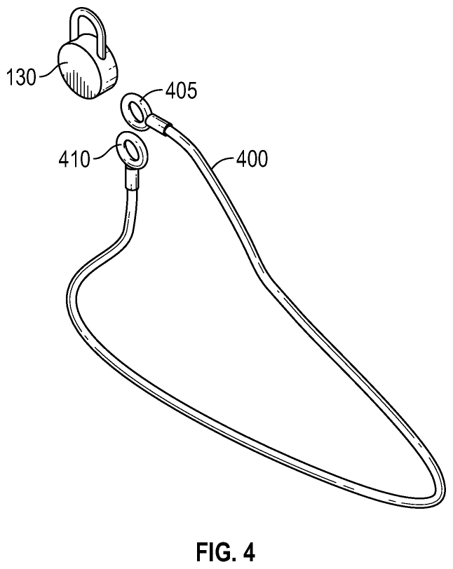

The lock may also include, as shown in FIG. 4, with a security cable 400, which can be a cable that is plastic or otherwise coated, having eyes 405, 410 at both ends. The cable can be wound around the object to be secured, thus preventing the object from being accessed until the lock is manually released.

In another embodiment, the wireless lock can be part of a different kind of locking mechanism.

An important feature of the system is its use as an interlock. An important feature of an interlock is that it automatically locks the car, every time the car is shut off. In order to carry out this function, at step 360, the app determines if the driving operation is complete. This can be done, for example, by the phone app automatically detecting whether the car is running or not. This can be done by wirelessly communicating with automobile systems, or by listening for the noise of a running automobile, or by detecting lack of movement for more than 5 minutes using its GPS, or by determining a distance from the phone to the vehicle.

If finished, the user is prompted to lock the vehicle, and to obtain a photo of the vehicle being locked. For example, this can be an image of the vehicle, showing the the cable being locked to the vehicle in a way that prevents someone from operating the vehicle until the lock has been removed.

If the system does not receive a picture of the vehicle being locked within a few minutes of detecting that the vehicle is shut off, then the user can be prompted, using the phone app, to lock the vehicle. If no locking evidence is provided after the prompting, an alarm is issued at 380, indicating that the interlock has not been operated. The alarm can alert law enforcement or can alert some other authority that the user has not properly relocked the vehicle.

As an alternative to obtaining a picture of the vehicle being locked, the system can detect the lock being closed, using the wireless capability of the lock. Of course, this is less secure, since the lock can be locked without locking it on to the vehicle, but is certainly an additional data point.

In an alternative embodiment, there can be locking eyelets installed onto the vehicle steering wheel or other vehicle part, and the lock can extend between those eyelets. The eyelets can also have a sensor that senses when the lock has been used to secure between the eyelets, thus automatically providing the vehicle locked detection.

The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A computer-based system for controlling access to a locked item, comprising:
an alcohol tester, receiving a user's breath, and producing an output indicative of an amount of alcohol in the user's breath,
the alcohol tester producing a wirelessly communicated output signal indicative of the amount of alcohol in the user's breath;
a wireless communicating lock, which receives an unlock command wirelessly, and unlocks responsive to receiving the unlock command; and
a portable device, running an application which wirelessly receives the output from the alcohol tester indicative of a concentration of alcohol in the user's breath,
and
where the portable device determines if the amount of alcohol in the user's breath is over a limit or under the limit, and responsive to determining that the concentration of alcohol is under the limit, sends the signal to the wireless communicating lock which unlocks the wireless communicating lock, and responsive to determining that the amount of alcohol in the user's breath is over the limit, does not send the signal to the lock and maintains the lock in the locked position.

2. The system as in claim 1, wherein the locked item is a locked vehicle.

3. The system as in claim 1, wherein the portable device is a mobile phone.

4. The system as in claim 1, where the application also controls obtaining a photograph of the user at a time when the user is using the alcohol tester.

5. The system as in claim 1, where the lock uses a flexible tamper proof chain.

6. The system as in claim 1, further comprising setting a level comprising the limit on a remote server, and allowing the level to be changed by a user who has sufficient credentials to change the level.

7. The system as in claim 4, wherein the application requires verifying the photograph of the user before sending the signal to the wireless communicating lock which unlocks the wireless communicating lock.

8. The system as in claim 7, wherein the portable device uses face identification to analyze the picture.

9. The system as in claim 1, wherein the application detects the vehicle being turned off, and responsive to the vehicle being turned off, prompts the user to provide evidence that the vehicle has been locked using the lock.

10. A method of using a computer-based system for controlling access to a locked item, comprising:
using an alcohol tester, receiving a user's breath, and producing an output indicative of an amount of alcohol in the user's breath,
producing a wirelessly communicated output signal indicative of the amount of alcohol in the user's breath;
running an application in a portable device, including a computing part, which wirelessly receives the output from the alcohol tester indicative of a concentration of alcohol in the user's breath,
and
determining if the amount of alcohol in the user's breath is over a limit or under the limit, and responsive to determining that the concentration of alcohol is under the limit, sends the signal to a wireless communicating lock which unlocks the wireless communicating lock, and responsive to determining that the amount of alcohol in the user's breath is over the limit, does not send the signal to the lock and maintains the lock in the locked position.

11. The method as in claim 10, wherein the locked item is a locked vehicle.

12. The method as in claim 10, wherein the portable device is a mobile phone.

13. The method as in claim 10, where the application also controls obtaining a photograph of the user, and requires verifying the photograph of the user before sending the signal to the wireless communicating lock which unlocks the wireless communicating lock.

14. The method as in claim 10, wherein the application requires obtaining a photograph of the user while the user is using the alcohol tester.

15. The method as in claim 10, wherein the application detects the vehicle being turned off, and responsive to the vehicle being turned off, prompts the user to provide evidence that the vehicle has been locked using the lock.
